(19) United States
(12) Patent Application Publication
(10) Pub. No.: US 2018/0286501 A1
(43) Pub. Date: Oct. 4, 2018

(54) Apparatus And Method For Facilitating Patient Identification In Conjunction With An Emergency Call

(71) Applicant: Nokia Technologies Oy, Espoo (FI)

(72) Inventors: Janne MARIN, Espoo (FI); Jarkko KNECKT, Espoo (FI); OIIi ALANEN, Vantaa (FI); Mika KASSLIN, Espoo (FI); Enrico-Henrik RANTALA, Berkeley, CA (US)

(21) Appl. No.: 15/765,410

(22) PCT Filed: Oct. 2, 2015
(86) PCT No.: PCT/IB2015/057566

§ 371 Date: Apr. 2, 2018

(51) Int. Cl. G16H 10/60; G06F 17/30

(52) U.S. Cl. CPC G16H 10/60; G06F 17/30864

(57) ABSTRACT

A method, apparatus and computer program product are provided for facilitating efficient patient identification in conjunction with an emergency call. In regards to a method, an emergency call from an intermediary device is received, and a search for one or more potential patient devices in proximity to an intermediary device is initialized. The method also includes receiving a unique identifier associated with a patient device from among the one or more potential patient devices. Further, the method retrieves medical information or medical record for a patient associated with the unique identifier. A corresponding apparatus and computer program product are also provided. Either Bluetooth Low Energy or WI-FI Aware are used. The mobility status comprising acceleration information of each patient device is transmitted.

### TECHNOLOGICAL FIELD

An example embodiment relates generally to patient identification and, more particularly, to a method, apparatus, and computer program product for facilitating efficient patient identification in conjunction with an emergency call.

### BACKGROUND

Emergency calls to an emergency dispatch center may be generated for various purposes. In medical emergency situations, it would be beneficial that patients receive medical assistance as soon as possible. The medical assistance that is rendered in an emergency situation may be most effective if the patient's medical history is taken into account. Thus, the patient identity and the resulting medical information detailing the patient's medical history could be helpful to the first responders administering first aid to the patient. However, if an accident victim is unconscious or otherwise unable to communicate, witnesses or volunteers with basic medical skills may be close by and willing to help. However, witnesses, volunteers or other first responders may lack crucial medical information about the victim's medical history and may not be able to treat the victim as well as is desired.

For individuals with ongoing medical conditions, patient tracking solutions have been developed. These patient tracking solutions may use global positioning system (GPS) or other location information to provide patient location information in conjunction with emergency calls. However, patient tracking solutions may provide low accuracy in a crowded location and, in any event, may provide only a limited amount, if any, medical information regarding the patient.

### BRIEF SUMMARY

A method, apparatus and computer program product are provided in accordance with an example embodiment in order to facilitate patient identification in conjunction with an emergency call. In an example embodiment, an intermediary device is provided in order to search one or more potential patient devices in proximity to the intermediary device. As such, the intermediary device may more reliably identify the patient device from among one or more potential patient devices in conjunction with an emergency call such that medical information relating to the patient may be provided in a timely manner to those responding to the emergency, thereby allowing the patient to be more effectively treated in a manner consistent with their medical history.

In an example embodiment, a method is provided that includes receiving an emergency call from an intermediary device. A method of this example embodiment also includes initializing a search for one or more potential patient devices in proximity to an intermediary device. The method of this example embodiment also includes receiving a unique identifier associated with a patient device from among the one or more potential patient devices. As such, medical information for a patient associated with the unique identifier may be retrieved.

The method of an example embodiment may identify the one or more potential patient devices in response to the search. The method of an example embodiment may also include causing an alarm message to be sent to each of the one or more potential patient devices. A method of this example embodiment also includes receiving an indication from the intermediary device of the patient device from among the one or more potential patient devices. In an example embodiment, the search is achieved by proximity radio capable services. In an example embodiment, the search is achieved by a local wireless network interface of WI-FI Aware (NAN), Bluetooth Low Energy (BLE), or Zigbee services.

The method of an example embodiment may apply a second filtering criteria to the one or more potential patient devices. In an example embodiment, the second filtering criteria comprises mobility status of the one or more potential patient devices. In an example embodiment, the mobility status comprises acceleration information of each of the one or more potential patient devices. The method of an example embodiment may also include decrypting the unique identifier to facilitate retrieval of the medical records of the patient. In an example embodiment, the unique identifier is an encrypted token.

In another example embodiment, a method is provided that includes initializing a search for one or more potential patient devices in proximity to an intermediary device. A method of this example embodiment also includes receiving an indication of the one or more potential patient devices in response to the search. The method of this example embodiment also includes identifying a patient device from the one or more potential patient devices for which the indication was received. As such, a unique identifier from the patient device that facilitates identification of a patient and retrieval of medical information associated with the patient may be received and then caused to be transmitted to a network device. In an example embodiment, the search is achieved by proximity radio capable services. In an example embodiment, the search is achieved by a local wireless network interface of WI-FI Aware (NAN), Bluetooth Low Energy (BLE), or Zigbee services.

The method of an example embodiment may apply a first filtering criteria to the one or more potential patient devices. In an example embodiment, the first filtering criteria comprises a range filter that filters the one or more potential patient devices within a predefined range from the intermediary device. The method of an example embodiment may also apply a second filtering criteria to the one or more potential patient devices. In an example embodiment, the second filtering criteria comprises mobility status of the one or more potential patient devices. In an example embodiment, the mobility status comprises acceleration information of each of the one or more potential patient devices. The method of an example embodiment may also decrypt the unique identifier to facilitate retrieval of the medical records of the patient. In an example embodiment, the unique identifier is an encrypted token.

In another example embodiment, an apparatus is provided that includes at least one processor and at least one memory including computer program code with the at least one memory and the computer program code configured to, with the processor, cause the apparatus to receive an emergency call from an intermediary device. The at least one memory and the computer program code are also configured to, with the processor, cause the apparatus of this example embodiment to initialize a search for one or more potential patient devices in proximity to an intermediary device. The at least one memory and the computer program code are also configured to, with the processor, cause the apparatus of this example embodiment to receive a unique identifier associated with a patient device from among the one or more potential patient devices. As such, medical information for a patient associated with the unique identifier may be retrieved.

The at least one memory and the computer program code may also be configured to, with the processor, cause the apparatus of an example embodiment to identify the one or more potential patient devices in response to the search. The at least one memory and the computer program code may also be configured to, with the processor, cause the apparatus of an example embodiment to cause an alarm message to be sent to each of the one or more potential patient devices. The at least one memory and the computer program code may also be configured to, with the processor, cause the apparatus of an example embodiment to receive an indication from the intermediary device of the patient device from among the one or more potential patient devices. In an example embodiment, the search is achieved by proximity radio capable services. In an example embodiment, the search is achieved by a local wireless network interface of WI-FI Aware (NAN), Bluetooth Low Energy (BLE), or Zigbee services.

The at least one memory and the computer program code may also be configured to, with the processor, cause the apparatus of an example embodiment to apply a second filtering criteria to the one or more potential patient devices. In an example embodiment, the second filtering criteria comprises mobility status of the one or more potential patient devices. In an example embodiment, the mobility status comprises acceleration information of each of the one or more potential patient devices. The at least one memory and the computer program code may also be configured to, with the processor, cause the apparatus of an example embodiment to decrypt the unique identifier to facilitate retrieval of the medical records of the patient. In an example embodiment, the unique identifier is an encrypted token.

In yet another example embodiment, an apparatus is provided that includes at least one processor and at least one memory including computer program code with the at least one memory and the computer program code configured to, with the processor, cause the apparatus to initialize a search for one or more potential patient devices in proximity to an intermediary device. The at least one memory and the computer program code are configured to, with the processor, cause the apparatus to receive an indication of the one or more potential patient devices in response to the search. The at least one memory and the computer program code are configured to, with the processor, cause the apparatus to identify a patient device from the one or more potential patient devices for which the indication was received. As such, a unique identifier from the patient device that facilitates identification of a patient and retrieval of medical information associated with the patient may be received and then caused to be transmitted to a network device. In an example embodiment, the search is achieved by proximity radio capable services. In an example embodiment, the search is achieved by a local wireless network interface of WI-FI Aware (NAN), Bluetooth Low Energy (BLE), or Zigbee services.

The at least one memory and the computer program code are configured to, with the processor, cause the apparatus of an example embodiment to apply a first filtering criteria to the one or more potential patient devices. In an example embodiment, the first filtering criteria comprises a range filter that filters the one or more potential patient devices within a predefined range from the intermediary device. The at least one memory and the computer program code are configured to, with the processor, cause the apparatus of an example embodiment to apply a second filtering criteria to the one or more potential patient devices. In an example embodiment, the second filtering criteria comprises mobility status of the one or more potential patient devices. In an example embodiment, the mobility status comprises acceleration information of each of the one or more potential patient devices. The at least one memory and the computer program code are configured to, with the processor, cause the apparatus of an example embodiment to decrypt the unique identifier to facilitate retrieval of the medical records of the patient. In an example embodiment, the unique identifier is an encrypted token.

In another example embodiment, a computer program product is provided that includes at least one non-transitory computer-readable storage medium having computer-executable program code portions stored therein with the computer-executable program code portions comprising program code instructions for receiving an emergency call from an intermediary device. The program code portions also comprise program code instructions for initializing a search for one or more potential patient devices in proximity to an intermediary device. The program code portions further comprise program code instructions for receiving a unique identifier associated with a patient device from among the one or more potential patient devices. As such, medical information for a patient associated with the unique identifier may be retrieved.

In yet another example embodiment, a computer program product is provided that includes at least one non-transitory computer-readable storage medium having computer-executable program code portions stored therein with the computer-executable program code portions comprising program code instructions for initializing a search for one or more potential patient devices in proximity to an intermediary device. The program code portions also comprise program code instructions for receiving an indication of the one or more potential patient devices in response to the search. The program code portions further comprise program code instructions for identifying a patient device from the one or more potential patient devices for which the indication was received. As such, a unique identifier from the patient device that facilitates identification of a patient and retrieval of medical information associated with the patient may be received and then caused to be transmitted to a network device. In an example embodiment, the search is achieved by proximity radio capable services. In an example embodiment, the search is achieved by a local wireless network interface of WI-FI Aware (NAN), Bluetooth Low Energy (BLE), or Zigbee services.

In yet another example embodiment, an apparatus is provided that includes means for receiving an emergency call from an intermediary device, means for initializing a search for one or more potential patient devices in proximity to an intermediary device, and means for receiving a unique identifier associated with a patient device from among the one or more potential patient devices. As such, the apparatus may also include means for retrieving medical information for a patient associated with the unique identifier.

In yet another example embodiment, an apparatus is provided that includes means for initializing a search for one or more potential patient devices in proximity to an intermediary device, means for receiving an indication of the one or more potential patient devices in response to the search, and means for identifying a patient device from the one or more potential patient devices for which the indication was received. As such, the apparatus may also include means for receiving and then causing to be transmitted to a network device, a unique identifier from the patient device that facilitates identification of a patient and retrieval of medical information associated with the patient.

### BRIEF DESCRIPTION OF THE DRAWINGS

Having thus described certain embodiments of the invention in general terms, reference will now be made to the accompanying drawings, which are not necessarily drawn to scale, and wherein:

FIG. 1 depicts a block diagram of a system that may be specifically configured in accordance with an example embodiment of the present invention;

FIG. 2 depicts a block diagram of an apparatus that may be specifically configured in accordance with an example embodiment of the present invention;

FIG. 3 depicts a block diagram of a system that illustrates communication between the various system components in accordance with an example embodiment of the present invention;

FIG. 4 depicts a diagram of a computing device that may be specifically configured in accordance with an example embodiment of the present invention;

FIG. 5 illustrates an example flowchart illustrating a method of operating an example apparatus in accordance with an embodiment of the present invention;

FIG. 6 illustrates an example flowchart illustrating a method of operating an example apparatus in accordance with an embodiment of the present invention; and

FIG. 7 depicts a signal flow diagram in accordance with an example embodiment of the present invention.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ refers to (a) hardware-only circuit implementations (e.g., implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

As defined herein, a “computer-readable storage medium,” which refers to a physical storage medium (e.g., volatile or non-volatile memory device), may be differentiated from a “computer-readable transmission medium,” which refers to an electromagnetic signal.

As used herein, an “intermediary device” is also known as a helper device and may include any device of witnesses or volunteers in proximity to the patient, regardless of whether the witness or volunteer who possesses the intermediary device places an emergency, e.g., 911, call. The intermediary device may not be capable of obtaining the patient's medical information locally or from the Internet. As another example, the term “intermediary device” as used herein may include any device that communicates between a server (for example, a remote cloud server or the emergency dispatch center) and the patient device carried by the patient.

As used herein, one or more “potential patient devices”, refers to one or more devices that are in proximity to both the patient and the intermediary device. As another example, the term “potential patient devices”, as used herein may include any device discovered by the intermediary device from a search via one or more proximity radio capable services (for example, WI-FI Aware (NAN), Bluetooth Low Energy (BLE), or Zigbee services). As another example, the term “potential patient devices”, as used herein may include any device that is manually managed or edited by the intermediary device or a server, including not only those that are automatically discovered via proximity-based services but also those manually added or otherwise identified.

As used herein, an “alarm message”, refers to an alarm signal such as a visual message, a light, a vibration, or a sound. As another example, the term “alarm message” as used herein may include plain text messaging with a unique identifier. As another example, the term “alarm message” as used herein may include an encrypted token.

As used herein, a “patient identification”, refers to any direct or indirect information associated with and serving to identify a patient. As another example, the term “patient identification” as used herein may include one or more unique identifiers, one or more serial numbers, one or more encrypted codes relating to the patient's name, one or more encrypted codes relating to the patient's identity, or one or more of the patient's medical records.

As used herein, a “WI-FI Aware (NAN)” refers to a system that uses Wi-Fi Aware™ technology. A Wi-Fi Aware™ system is a Wi-Fi Alliance certification program configured to ensure interoperability of devices that implement features specified in the Wi-Fi Neighbor Awareness Networking (NAN) technical specification that has been developed by the Wi-Fi Alliance. The NAN provides a technique for discovery of services, information, etc. that can be run continuously in the background of a device without a significant effect on standby time of the device. The NAN may make all or near all of the NAN capable devices that have the NAN stack active available at the same time in the same channel for discovery. Common time periods are called NAN discovery windows and happen approximately every 0.5 seconds.

Bluetooth Low Energy (BLE) (a.k.a Bluetooth Smart) is a Bluetooth version configured for low power operations. A device that supports BLE may be configured to be discoverable and to offer services for other devices close by using advertising to indicate presence and availability of itself and/or its' services. An advertising device may transmit advertising channel packets on dedicated advertising channels and may listen to and respond to responses triggered by the advertising packets. A device may also act as a scanner or an initiator. When scanning, a device may look for advertising devices and may request further information from detected advertising devices. A device may operate as an initiator when establishing or attempting to establish a connection with another device. An initiator may request a connection with an advertising device upon receiving an advertising packet which allows a connection request.

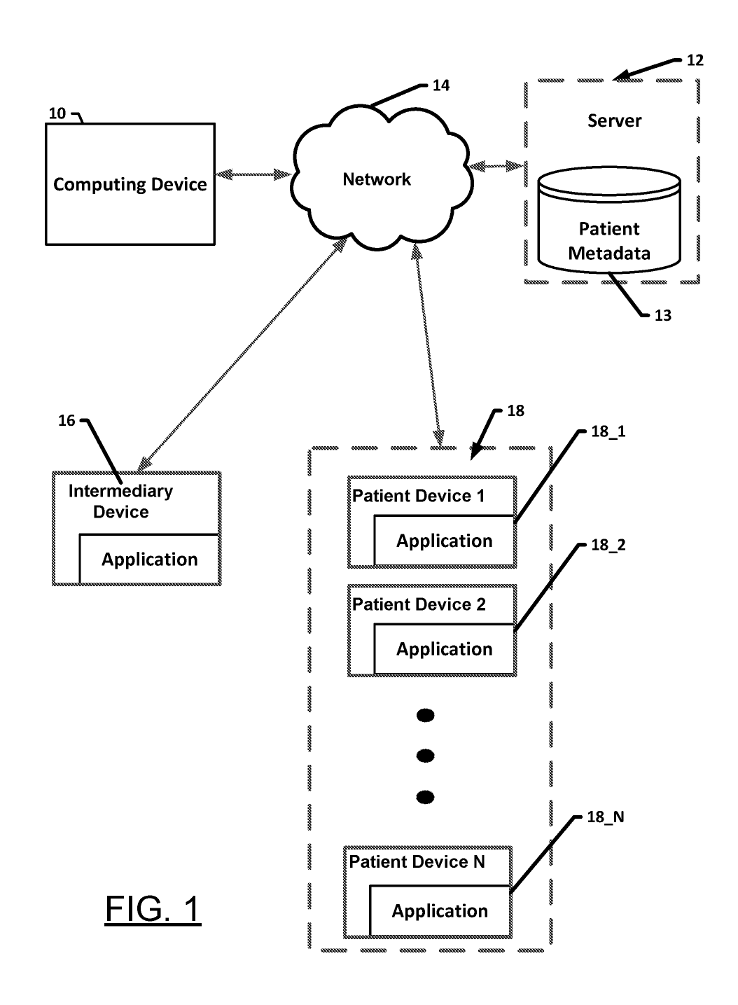

Referring now to FIG. 1, a system that supports communication, either wirelessly or via a wireline, between a computing device 10, an intermediary device 16, one or more potential patient devices 18 (18_1-18_N) and server 12 or other network entity (hereinafter generically referenced as a “server”) is illustrated. As shown, the computing device 10, the intermediary device 16, the one or more patient device 18 and the server 12 may be in communication via a network 14, such as a wide area network, such as a cellular network or the Internet, or a local area network such as a local wireless network (e.g., (NAN), BLE, or Zigbee). However, the computing device 10, the intermediary device 16, the one or more patient device 18 and the server 12 may be in communication in other manners, such as via direct communications between the computing device and the server or communications through an application that runs on both of the computing device 10, the intermediary device 16, the one or more potential patient devices 18, the server 12 or other network entity. The computing device 10, the intermediary device 16, and the one or more patient devices 18 may be hereinafter described as mobile devices and/or mobile terminals, but may be either mobile or fixed in the various embodiments.

The computing device 10, the intermediary device 16, and the one or more patient device 18 may be embodied by a number of different devices including mobile computing devices, such as a personal digital assistant (PDA), mobile telephone, smartphone, laptop computer, tablet computer, or any combination of the aforementioned, and other types of voice and text communications systems. Alternatively, the computing device 10 may be a fixed computing device, such as a personal computer, a computer workstation or the like. The server 12 may comprise storage devices for medical information, including, for example, Patient Metadata 13 as shown in FIG. 1. The server 12 may also be embodied by a computing device and, in one embodiment, is embodied by a web server. Additionally, while the system of FIG. 1 depicts a single server, the server may be comprised of a plurality of servers which may collaborate to support browsing activity, including that conducted by the computing device 10.

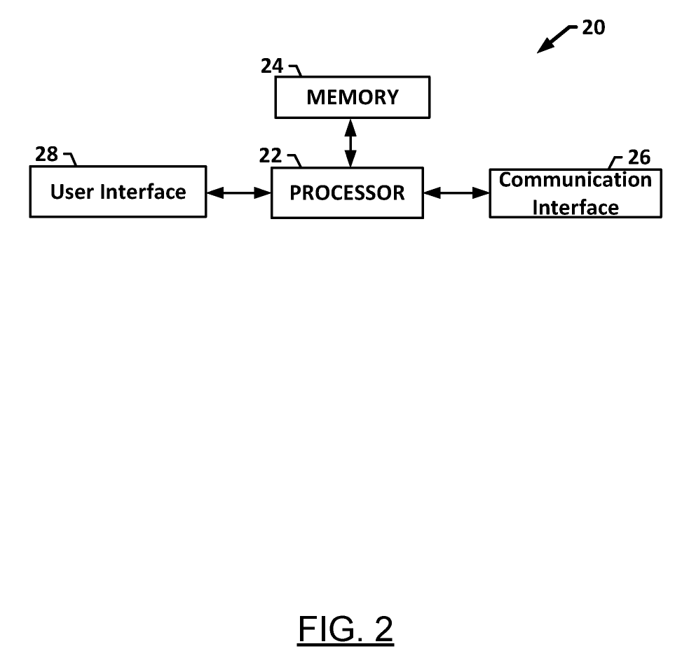

Regardless of the type of devices that embody the computing device 10, the intermediary device 16 and the one or more patient devices 18, the computing device 10, the intermediary device and the one or more patient devices may each include or be associated with an apparatus 20 as shown in FIG. 2. In this regard, the apparatus may include or otherwise be in communication with a processor 22, a memory device 24, a communication interface 26 and a user interface 28. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

In some embodiments, the processor 22 (and/or co-processors or any other processing circuitry assisting or otherwise associated with the processor) may be in communication with the memory device 24 via a bus for passing information among components of the apparatus. The memory device may include, for example, one or more volatile and/or non-volatile memories. In other words, for example, the memory device may be an electronic storage device (e.g., a computer readable storage medium) comprising gates configured to store data (e.g., bits) that may be retrievable by a machine (e.g., a computing device like the processor). The memory device may be configured to store information, data, content, applications, instructions, or the like for enabling the apparatus 20 to carry out various functions in accordance with an example embodiment of the present invention. For example, the memory device could be configured to buffer input data for processing by the processor. Additionally or alternatively, the memory device could be configured to store instructions for execution by the processor.

As noted above, the apparatus 20 may be embodied by a computing device 10, an intermediary device 16 and/or one or more patient devices 18 configured to employ an example embodiment of the present invention. However, in some embodiments, the apparatus may be embodied as a chip or chip set. In other words, the apparatus may comprise one or more physical packages (e.g., chips) including materials, components and/or wires on a structural assembly (e.g., a baseboard). The structural assembly may provide physical strength, conservation of size, and/or limitation of electrical interaction for component circuitry included thereon. The apparatus 20 may therefore, in some cases, be configured to implement an embodiment of the present invention on a single chip or as a single “system on a chip.” As such, in some cases, a chip or chipset may constitute means for performing one or more operations for providing the functionalities described herein.

The processor 22 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor 22 may be configured to execute instructions stored in the memory device 24 or otherwise accessible to the processor. Alternatively or additionally, the processor may be configured to execute hard coded functionality. As such, whether configured by hardware or software methods, or by a combination thereof, the processor may represent an entity (e.g., physically embodied in circuitry) capable of performing operations according to an embodiment of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (e.g., a head mounted display) configured to employ an embodiment of the present invention by further configuration of the processor by instructions for performing the algorithms and/or operations described herein. The processor may include, among other things, a clock, an arithmetic logic unit (ALU) and logic gates configured to support operation of the processor. In one embodiment, the processor may also include user interface circuitry configured to control at least some functions of one or more elements of the user interface 28.

Meanwhile, the communication interface 26 may be any means such as a device or circuitry embodied in either hardware or a combination of hardware and software that is configured to receive and/or transmit data between the computing device 10 or intermediary device 16 and a server 12 or between the intermediary device and one or more patient devices 18. In this regard, the communication interface 26 may include, for example, an antenna (or multiple antennas) and supporting hardware and/or software for enabling communications wirelessly. Additionally or alternatively, the communication interface may include the circuitry for interacting with the antenna(s) to cause transmission of signals via the antenna(s) or to handle receipt of signals received via the antenna(s). For example, the communications interface may be configured to communicate wirelessly, such as via Wi-Fi, Bluetooth or other wireless communications techniques. In some instances, the communication interface may alternatively or also support wired communication. As such, for example, the communication interface may include a communication modem and/or other hardware/software for supporting communication via cable, digital subscriber line (DSL), universal serial bus (USB) or other mechanisms.

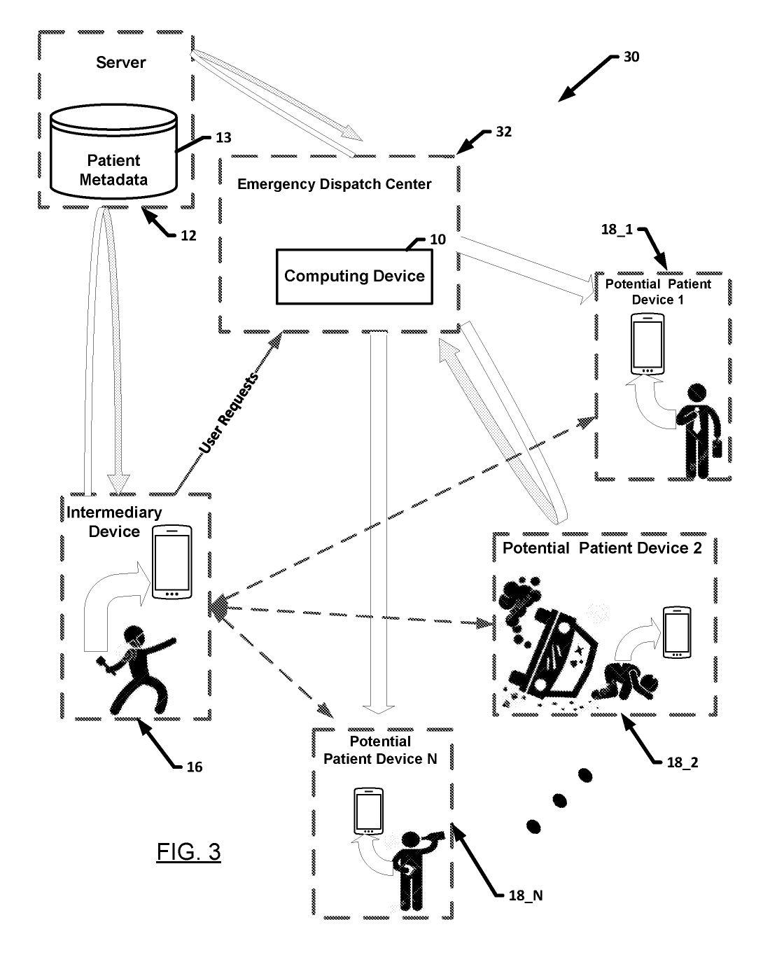

Referring now to FIG. 3, an example system is depicted illustrating a method, apparatus and computer program product in accordance with an embodiment of the present invention. As shown, the intermediary device 16 and the one or more patient devices 18 may be in communication via a local wireless network (e.g., (NAN), BLE, or Zigbee). The intermediary device 16 and the one or more patient devices 18 may also be in communication with the server 12 and the emergency dispatch center 32 via a wide area network (e.g., a cellular network or the Internet). The patient's medical information (e.g., patient's identity, patient's unique identifier and patient's medical information) may be exchanged between the emergency dispatch center 32 and the server 12, as well as between the intermediary device 16 and the emergency dispatch center 32. In one embodiment, the intermediary device 16 may grant access to the server and exchange information directly. In another embodiment, a potential patient device 18 may communicate with the emergency dispatch center and send or receive information.

The computing device 10 may be embodied by the server 12, the intermediary device 16 and/or the emergency dispatch center 32. Alternatively, the computing device 10 may be separate from and in communication with the server 12, the intermediary device 16 and the emergency dispatch center 32 as shown in FIG. 3. Additionally, while the system of FIG. 3 depicts a single emergency dispatch center 32 that includes the computing device 10, the system of another example embodiment may include a plurality of emergency dispatch centers 32 which may collaborate to support browsing activity conducted by the computing device 10 (for example, retrieving patient's medical information).

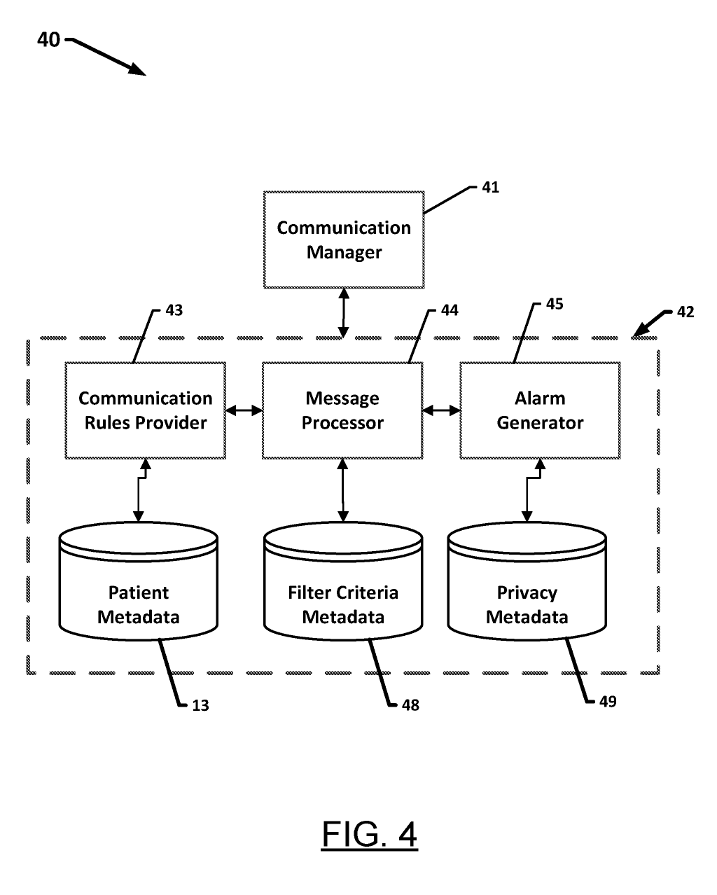

Referring now to FIG. 4, which is a block diagram illustrating a computer platform structure 40 embodied by the computing device 10 in accordance with an embodiment of the present invention. The computer platform structure 40 includes a communication manager 41 interacting with a metadata directory 42. The metadata directory 42 may include a communication rules provider 43, a message processor 44, and an alarm generator 45. The metadata directory 42 may also maintain metadata that is used by the method, apparatus and computer program product in accordance with an embodiment of the present invention. Patient metadata 13 may comprise patient information, for example, patient's identity, patient's unique identifier, or patient's medical information. Filter criteria metadata 48 may include a range filter that filters the one or more potential patient devices within a predefined range from the intermediary device 16. Additionally or alternatively, the filter criteria metadata 48 may include mobility status (e.g. acceleration information) of the one or more potential patient devices. Privacy metadata 49 may limit the availability of the patient metadata 13. In the example embodiment of FIG. 4, the patient metadata 13, the filter criteria metadata 48 and the privacy metadata 49 may be embodied by the memory 24 of FIG. 2, while the other components of FIG. 4 including the communication manager 41, the communication rules provider 43, the message processor 44 and alarm generator 45 are embodied by the processor 22.

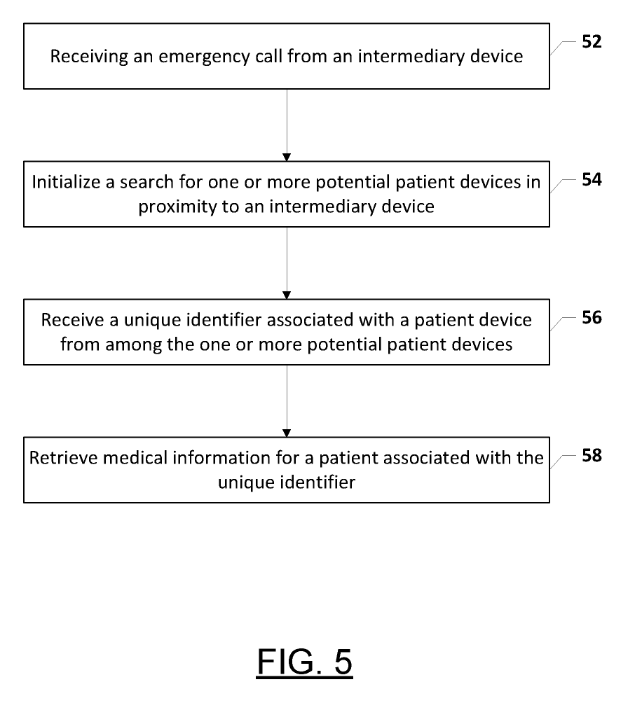

FIG. 5 is an example flowchart illustrating a method for facilitating patient identification in conjunction with an emergency call in accordance with an embodiment of the present invention. The operations of FIG. 5 may be performed by a network device, such as the server 12 or emergency call center 32, which embodies an apparatus 20. In some embodiments, the operations of FIG. 5 are not limited to any particular network (e.g., cellular systems). For example, non-cellular solutions such as a wireless local area network (WLAN) (e.g., (NAN), BLE, or Zigbee services) may be utilized for at least some of the operations, such as to permit the facilitation of the proximity retrieval process of medical information associated with the patient described in FIG. 5.

As such, as shown in block 52 of FIG. 5, apparatus 20 may be configured to receive an emergency call, such as from an intermediary device 16 or another device, reporting a patient in need of emergency, e.g., medical, services. The apparatus therefore includes means, such as the processor 22, the communication interface 26 or the like, for receiving an emergency call, such as from an intermediary device.

As shown in block 54 of FIG. 5, the apparatus 20 may be configured to initialize a search for one or more potential patient devices in proximity to an intermediary device. The apparatus therefore includes means, such as the processor 22, the communication interface 26 or the like, for initializing a search for one or more potential patient devices in proximity to an intermediary device. The search may be initialized in various manners, but, in one example embodiment, the apparatus embodied by a network device is configured to communicate with the intermediary device and to cause or instruct the intermediary device to identify the potential patient devices in proximity to the intermediary device, such as by (NAN), BLE, or Zigbee services. Since the patient that is the subject of the emergency call is proximate the intermediary device, the patient device, such as a smart phone, a health monitoring device, etc. carried by the patient, is also proximate the intermediary device and should respond to the query transmitted by the intermediary device (along with the communication devices carried by other people who are proximate to the intermediary device).

In some embodiments, the apparatus 20 also includes means, such as the processor 22, the communication interface 26 or the like, for identifying the one or more potential patient devices in response to the search. In this regard, the apparatus may receive, from the intermediary device, an indication of the one or more potential patient devices in response to the query by the intermediary device. The apparatus of this example embodiment also includes means, such as the processor 22, the communication interface 26 or the like, for causing an alarm message to be sent to each of the one or more potential patient devices, such as by communicating directly with each potential patient device or by instructing the intermediary device to cause an alarm message to be sent to each potential patient device. The apparatus of this example embodiment also includes means, such as the processor 22, the communication interface 26 or the like, for receiving an indication from the intermediary device of the patient device from among the one or more potential patient devices. For example, the potential patient devices may be caused to sequentially generate an alert and the intermediary device may identify the patient device in response to its generation of the alarm. In some embodiments, the apparatus embodied by intermediary device 16 therefore includes means, such as the processor 22, the communication interface 26 or the like, for obtaining further filtering rules from the emergency dispatch center 32 to filter down the one or more potential patient devices.

As shown in block 56 of FIG. 5, an apparatus 20 may be configured to receive a unique identifier associated with a patient device from among the one or more potential patient devices. The apparatus therefore includes means, such as the processor 22, the communication interface 26 or the like, for receiving a unique identifier associated with a patient device from among the one or more potential patient devices. For example, the unique identifier may be received from the intermediary device which, in turn, received the unique identifier from the patient device. Alternatively, the unique identifier may be obtained from a database that associates patient devices with respective unique identifiers.

As such, as shown in block 58 of FIG. 5, an apparatus 20 may also be configured to retrieve medical information for a patient associated with the unique identifier. The apparatus therefore includes means, such as the processor 22, the communication interface 26 or the like, for retrieving medical information for a patient associated with the unique identifier, such as from a database. By relaying the medical information to a care professional who will treat the patient, the patient can be treated in a more informed and effective manner.

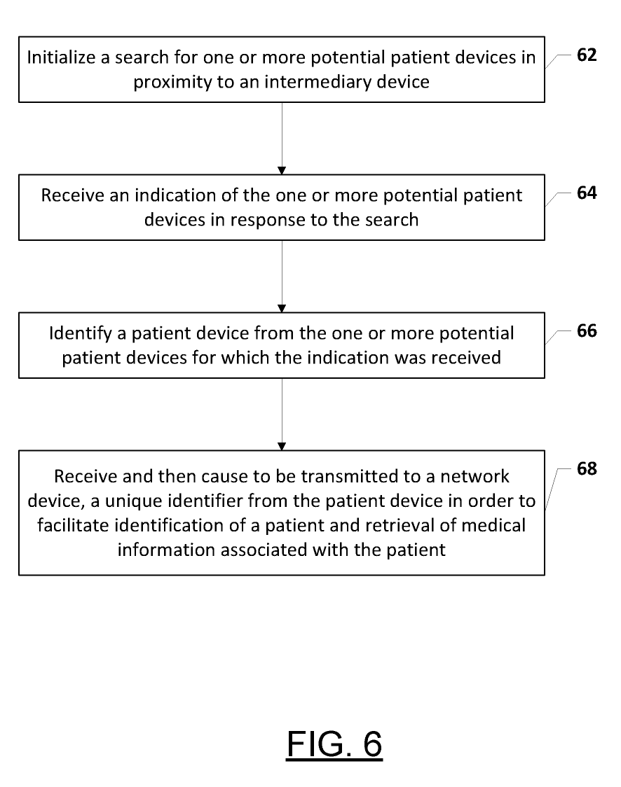

FIG. 6 is an example flowchart illustrating a method for facilitating patient identification in accordance with an embodiment of the present invention. The operations of FIG. 6 may be performed by an intermediary device 16, such as may be embodied by a smart phone, for example. In some embodiments, the operations of FIG. 6 are not limited to any particular network (e.g., cellular systems). For example, non-cellular solutions such as a wireless local area network (WLAN) (e.g., (NAN), BLE, or Zigbee services) may similarly permit the facilitation of the proximity retrieval process of medical information associated with the patient described in FIG. 6. Moreover, the operations of FIG. 6 may be performed by any other computing device, such as a laptop, tablet, or desktop computer or the like.

As such, as shown in block 62 of FIG. 6, an apparatus 20 embodied by the intermediary device 16 may be configured to initialize a search for one or more potential patient devices in proximity to an intermediary device. The apparatus embodied by intermediary device 16 therefore includes means, such as the processor 22, the communication interface 26 or the like, for initializing a search for one or more potential patient devices in proximity to an intermediary device, such as in response to the user of the intermediary device observing the patient, in response to an instruction from a network entity or following placement of an emergency call. One or more proximity radio capable services (for example, (NAN), BLE, or Zigbee services) may be utilized in the search.

As shown in block 64 of FIG. 6, an apparatus 20 embodied by the intermediary device 16 may be configured to receive an indication of the one or more potential patient devices in response to the search. The apparatus embodied by intermediary device 16 therefore includes means, such as the processor 22, the communication interface 26 or the like, for receiving an indication of the one or more potential patient devices in response to the search. In this regard, the apparatus may receive, from the one or more potential patient devices, an indication of the one or more potential patient devices, such as a reply message from the potential patient device(s), in response to the query by the intermediary device. The apparatus of this example embodiment may also include means, such as the processor 22, the communication interface 26 or the like. In some embodiments, the apparatus embodied by intermediary device 16 therefore includes means, such as the processor 22, the communication interface 26 or the like, for obtaining further filtering rules from the emergency dispatch center 32 to filter down the one or more potential patient devices.

As shown in block 66 of FIG. 6, an apparatus 20 embodied by the intermediary device 16 may be configured to identify a patient device from the one or more potential patient devices for which the indication was received. The apparatus embodied by intermediary device 16 therefore includes means, such as the processor 22, the communication interface 26 or the like, for identifying a patient device from the one or more potential patient devices for which the indication was received. For example, the apparatus of this example embodiment may include means, such as the processor 22, the communication interface 26 or the like, for causing an alarm message to be sent to each of the one or more potential patient devices, such as by communicating directly with each potential patient device. The potential patient devices may therefore be caused to sequentially generate an alert and the intermediary device may identify the patient device in response to its generation of the alarm. The alert may including a visual message, a light, a vibration, or a sound. In addition or alternatively, the apparatus may identify the patient device from among the one or more potential patient devices in other manners including through filtering of the indications from the potential patient device(s) based upon a distance threshold (e.g. less than two meters), such as provided by the filter criteria metadata 48, between the intermediary device and the one or more potential patient devices in instances in which the intermediary device is close to the patient. Still further, the apparatus may additionally or alternatively identify the patient device based upon the mobility status, such as acceleration data, from the potential patient devices including, for example, an indication as to whether or not a potential patient device is currently in motion. As such, the intermediary device of an example embodiment may identify the patient device from among the one or more potential patient devices by initially identifying the potential patient device(s) that are both within the distance threshold and stationary (in an instance in which the patient is lying still) and then causing those nearby devices to generate an alert such that the intermediary device may confirm the patient device based upon the alert generated thereby.

As such, as shown in block 68 of FIG. 6, an apparatus 20 embodied by the intermediary device 16 may be configured to receive and then cause to be transmitted to a network device, such as the server 12 or other network device, a unique identifier from the patient device that facilitates identification of a patient and retrieval of medical information associated with the patient. The apparatus embodied by user device 16 therefore includes means, such as the processor 22, the communication interface 26 or the like, for receiving and then causing to be transmitted to a network device, a unique identifier from the patient device that facilitates identification of a patient and retrieval of medical information associated with the patient.

The unique identifier may be encrypted and/or may be in the form of a token. In an example embodiment, the apparatus 20 embodied by intermediary device 16 therefore includes means, such as the processor 22, the communication interface 26 or the like, for receiving and transmitting an encrypted identifier and/or token to a network device. In this regard, the intermediary device 16 may utilize the encrypted identifier and/or token to identify the patient, while the confidentiality of the patient is maintained with the identifier being encrypted until it reaches a trusted party, such as the network device.

FIGS. 5 and 6 illustrate flowcharts of an apparatus, method and computer program product according to example embodiments of the invention. FIG. 5 is shown from the perspective of the network device, while FIG. 6 is from the perspective of the intermediary device 16. It will be understood that each block of the flowcharts, and combinations of blocks in the flowchart, may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory device 24 of an apparatus employing an embodiment of the present invention and executed by a processor 22 of the apparatus. It should be appreciated though, that in the various embodiments in which the apparatus 20 is embodied by the computing system of FIG. 4 that the operations illustrated in FIGS. 5 and 6 may be performed by various system components in the computing system of FIG. 4. For example, the operations performed by the processor 22 may be performed by components of the embodiment of FIG. 4 including the communication manager 41, the communication rules provider 43, the message processor 44 and alarm generator 45 and the operations performed by the memory 24 may utilize the patient metadata 13, the filter criteria metadata 48 and the privacy metadata 49. As will be appreciated, any such computer program instructions may be loaded onto a computer or other programmable apparatus (for example, hardware) to produce a machine, such that the resulting computer or other programmable apparatus implements the functions specified in the flowchart block(s). These computer program instructions may also be stored in a non-transitory computer-readable memory that may direct a computer or other programmable apparatus to function in a particular manner, such that the instructions stored in the computer-readable memory produce an article of manufacture the execution of which implements the function specified in the flowchart block(s). The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart block(s). As such, the operations of FIGS. 5 and 6, when executed, convert a computer or processing circuitry into a particular machine configured to perform an example embodiment of the present invention. Accordingly, the operations of FIGS. 5 and 6 define an algorithm for configuring a computer or processing to perform an example embodiment. In some cases, a general purpose computer may be provided with an instance of the processor which performs the algorithms of FIGS. 5 and 6 to transform the general purpose computer into a particular machine configured to perform an example embodiment.

Accordingly, blocks of the flowcharts support combinations of means for performing the specified functions and combinations of operations for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

In some embodiments, certain ones of the operations above may be modified or further amplified. Moreover, in some embodiments additional optional operations may also be included as shown by the blocks having a dashed outline in FIGS. 5 and 6. It should be appreciated that each of the modifications, optional additions or amplifications below may be included with the operations above either alone or in combination with any others among the features described herein.

In some example embodiments, a method, apparatus and computer program product may be configured for facilitating/enabling efficient patient identification in conjunction with an emergency call, and more specifically, for facilitating/enabling efficient patient identification in conjunction with an emergency call using a user device such as a smart phone, cellular phone, tablet, laptop or any type of mobile device.

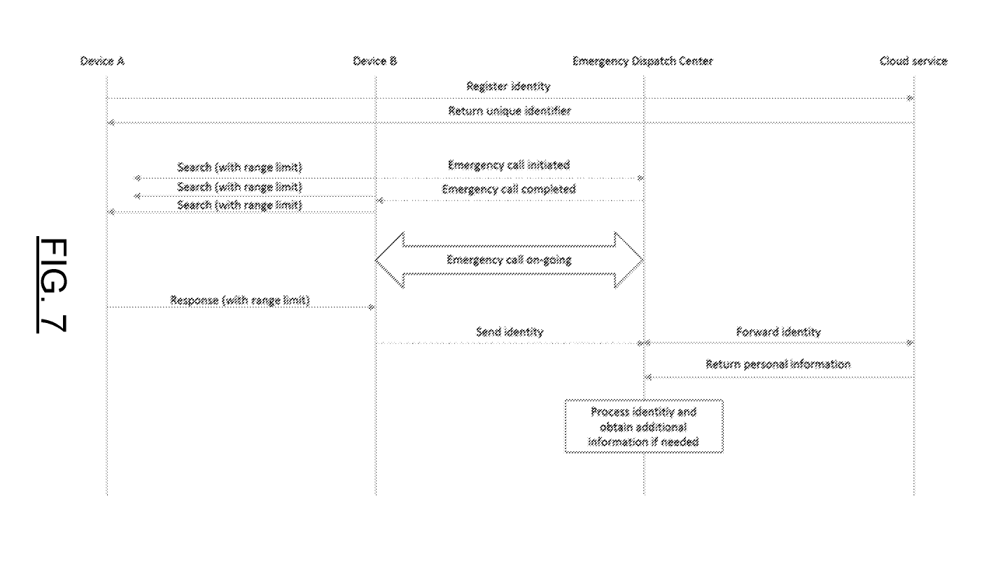

Referring now to FIG. 7, the signaling associated with a method, apparatus and computer program product in accordance with an embodiment of the present invention is illustrated. In this example, Device A is the patient device and device B is the intermediary device. Device A may be registered to the cloud service. In one embodiment, a search is initiated automatically when an emergency call is made through Device B. Next, Device A receives a request from Device B, and responds the request by sending patient's unique identifier to Device B. Device B forwards the patient's unique identifier to the emergency dispatch center. In one embodiment, application of service handing the search and connecting to the cloud service may require a periodic change in the service name or ID to protect patient's identity. Based on the unique identifier, the emergency dispatch center or other trusted network device may obtain medical information for the patient and provide the medical information to a care professional who, in turn, can better care for the patient.

As shown in the example embodiment of FIG. 7, each patient device (designated as Device A) initially registers its identify with a network device, such as represented by the cloud service, which, in turn, provides a unique identifier that may be stored by the patient device. In response to an emergency call, such as from an intermediary device (designated as Device B) to the emergency dispatch center, the intermediary device may identify potential patient devices by searching within a predefined range limit of distances. From the responses from the potential patient devices, the intermediary device identifies the identifier of the patient device which, in turn, is forwarded to the emergency dispatch center and the network device. As such, the network device may provide medical information for the patient to the emergency dispatch center to facilitate treatment of the patient. In one embodiment, the range limit may be shortened by using multiple “search with range limit” messages initiated from Device B to Device A. By way of example, in order to filter down the number of potential patients, a first message may try to find potential patients within 20 meters of Device B, a second message may try to find potential patients within 10 meters, and a third message may try to find potential patients within 2 meters. It should be appreciated that different distances and/or different combinations of multiple distances may be used in each of the multiple messages. In one embodiment, the multiple messages have the same content in order to add redundancy and make the search more reliable. In some embodiments, Device B may obtain further filtering rules from the emergency dispatch center to filter down the number of potential patients.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A method comprising:
receiving an emergency call from an intermediary device;
initializing a search for one or more potential patient devices in proximity to the intermediary device;
receiving a unique identifier associated with a patient device from among the one or more potential patient devices; and
retrieving medical information for a patient associated with the unique identifier.

2. The method of claim 1, further comprising
identifying the one or more potential patient devices in response to the search;
causing an alarm message to be sent to each of the one or more potential patient devices; and
receiving an indication from the intermediary device of the patient device from among the one or more potential patient devices.

3. The method of claim 1, wherein the search is achieved by a local wireless network interface of WI-FI Aware (NAN), Bluetooth Low Energy (BLE), or Zigbee services.

4. The method of claim 1, further comprising applying a first filtering criteria to the one or more potential patient devices, wherein the first filtering criteria comprises a range filter that filters the one or more potential patient devices within a predefined range from the intermediary device.

5. The method of claim 1, further comprising applying a second filtering criteria to the one or more potential patient devices, wherein the second filtering criteria comprises mobility status of the one or more potential patient devices.

6. The method of claim 5, wherein the mobility status comprises acceleration information of each of the one or more potential patient devices.

7. The method of claim 1, further comprising decrypting the unique identifier to facilitate retrieval of the medical records of the patient, wherein the unique identifier is an encrypted token.

8-20. (canceled)

21. An apparatus comprising at least one processor and at least one memory including computer program code, the at least one memory and the computer program code configured to, with the processor, cause the apparatus to at least:
initialize a search for one or more potential patient devices in proximity to the apparatus;
receive an indication of the one or more potential patient devices in response to the search;
identify a patient device from the one or more potential patient devices for which the indication was received; and
receive and then cause to be transmitted to a network device, a unique identifier from the patient device that facilitates identification of a patient and retrieval of medical information associated with the patient.

22. The apparatus of claim 21, wherein the search is achieved by a local wireless network interface of WI-FI Aware (NAN), Bluetooth Low Energy (BLE), or Zigbee services.

23. The apparatus of claim 21, wherein the at least one memory and the computer program code are configured to, with the processor, cause the apparatus to apply a first filtering criteria to the one or more potential patient devices, wherein the first filtering criteria comprises a range filter that filters the one or more potential patient devices within a predefined range from the apparatus.

24. The apparatus of claim 21, wherein the at least one memory and the computer program code are configured to, with the processor, cause the apparatus to apply a second filtering criteria to the one or more potential patient devices, wherein the second filtering criteria comprises mobility status of the one or more potential patient devices.

25. The apparatus of claim 24, wherein the mobility status comprises acceleration information of each of the one or more potential patient devices.

26. The apparatus of claim 21, wherein the at least one memory and the computer program code are further configured to, with the processor, cause the apparatus to decrypt the unique identifier to facilitate retrieval of the medical records of the patient, wherein the unique identifier is an encrypted token.

27. A computer program product comprising at least one non-transitory computer-readable storage medium having computer-executable program code portions stored therein, the computer-executable program code portions comprising program code instructions for performing the method of claim 1.

28. (canceled)
